(19) United States
(12) Patent Application Publication
(10) Pub. No.: US 2023/0273110 A1
(43) Pub. Date: Aug. 31, 2023

(54) METHODS AND SYSTEM FOR VALIDATING FLOW CYTOMETRY MEASUREMENTS

(71) Applicant: KIADIS PHARMA INTELLECTUAL PROPERTY B.V., Amsterdam (NL)

(72) Inventor: Kam-Wing LING, Amsterdam (NL)

(21) Appl. No.: 17/919,708

(22) PCT Filed: Apr. 21, 2021
(86) PCT No.: PCT/IB2021/053301

§ 371 Date: Oct. 18, 2022

Related U.S. Application Data
(60) Provisional application No. 63/013,098, filed on Apr. 21, 2020.

(51) Int. Cl. G01N 15/14; G01N 33/569

(52) U.S. Cl. CPC G01N 15/1434; G01N 33/56972; G01N 2015/0065

(57) ABSTRACT

Disclosed are validation methods and systems for characterizing the credibility and reproducibility of measurements made by a fluorescence-based analytical instrument.

### CROSS-REFERENCE TO RELATED APPLICATIONS

This application is a U.S. National Stage application of PCT/IB2021/053301 filed on Apr. 21, 2021, which claims the benefit of priority to U.S. Provisional Application No. 63/013,098 filed on Apr. 21, 2020, the entire contents of which is herein incorporated by reference.

### FIELD

The present disclosure relates to fluorescence-based analytical methods and systems, and particularly to validation methods to evaluate the credibility and reproducibility of measurements by a fluorescence-based analytical instrument.

### BACKGROUND

Fluorescence-based cellular analysis systems such as flow cytometry and fluorescence activated cell sorting (FACS) are powerful tools for measuring various cellular elements in a test sample, such as cell surface receptors and intracellular components. In FACS methods, antibody-conjugated fluorochromes (fluorescent dyes) are used to stain samples, and thereby signal the presence and amount of elements such as cellular receptors or other cell markers, peptides, and nucleic acids. The development of multiple different fluorochromes each having a unique, identifiable emission spectrum allows the simultaneous use of more than one fluorochrome at a time. As a consequence of their combined power and versatility, flow cytometry and FACS are commonly employed throughout drug development process, for example for immunophenotyping, receptor expression or occupancy, and other functional assays.

While powerful, flow cytometry and FACS methods are generally more challenging to validate than other analytical methods, for a variety of reasons. Among these reasons is the lack of protocol standardization, lack of standardized reference materials, and the use of complex and sensitive instrumentation. The monoclonal and polyclonal antibodies typically used for staining are not standardized, and can vary substantially in quality and performance. Thus, it is incumbent on the researcher to carefully assess that the reagents being used are working as intended. Yet the researcher has little guidance for performing validation of a given method and instrument. According to the US FDA for example, validation is simply defined as an evaluation of the method on its fitness for the intended applications, and recommends that a validation testing process must ensure that the assay meets predetermined standards and performs reliably and is fit for its intended use. The FDA guidelines acknowledge that no one-size-fits-all validation regulation is appropriate, and do not provide specific guidance for validating assays. The absence of standard or accepted regulatory guidelines and reference materials for validation is a challenge unfortunately not well addressed by the literature. The challenge of method validation is even greater when a target cell or marker of interest in a sample is believed to be present at very low concentrations. A need exists for improved validation methods for validating the performance of fluorescence-based analytical methods and systems.

### SUMMARY OF INVENTION

Among the various aspects of the present disclosure is a method for validating measurements by a fluorescence-based analytical instrument of a stained target cell population in a test sample, the method comprising: (a) negatively staining or having negatively stained cells in a first portion of a reference sample comprising target cells expressing a target cell marker; (b) positively staining or having positively stained the target cells in a second portion of the reference sample; (c) running the reference sample first portion through the instrument to obtain a fluorescence measurement indicative of the target cell concentration in the reference sample first portion, and running the reference sample second portion through the instrument to obtain a fluorescence measurement indicative of the target cell concentration in the reference sample second portion; (d) based on the fluorescence measurements obtained in (c), preparing a dilution series comprising a plurality of dilution samples, each dilution sample having a nominal concentration of the target cells, each nominal cell concentration greater than the concentration of target cells indicated by the fluorescence measurement of the negatively stained first portion in (a), wherein the nominal concentration in each dilution sample differs from the nominal concentration in each of the remaining dilution samples; (e) running the series of dilution samples from (d) through the instrument to obtain a series of fluorescence measurements comprising a fluorescence measurement for each a dilution sample; and (f) for each dilution sample, comparing the nominal cell concentration from (d) and the fluorescence measurement from (e) to quantify the performance of the staining method in the instrument. The instrument may be for example, a flow cytometer.

In another aspect, the present disclosure provides a method for validating measurements by a flow cytometer of a stained target cell population in a test sample, the method comprising: (a) negatively staining or having negatively stained cells in a first portion of a reference sample comprising target cells expressing a target cell marker; (b) positively staining or having positively stained the target cells in a second portion of the reference sample; (c) running both the reference sample first portion and second portion through the flow cytometer to obtain fluorescence measurements indicative of the target cell concentration in each of the reference sample first portion and the reference sample second portion; (d) based on the fluorescence measurements obtained in (c), preparing a series of dilution samples each having a nominal concentration of the target cells varying systematically across the dilution series, wherein the nominal cell concentration of at least one dilution sample is greater than the concentration of target cells indicated by the fluorescence measurement of the negatively stained first portion in (a); (e) running the series of dilution samples from (d) through the flow cytometer to obtain a series of fluorescence measurements comprising a fluorescence measurement for each a dilution sample; and (f) for each dilution sample, comparing the nominal cell concentration from (d) and the fluorescence measurement from (e) to quantify the performance of the staining method in the flow cytometer.

Any of the disclosed methods may comprise one or more of any of the following features. The comparing in (f) may comprise, for example, performing a statistical calculation on the difference between the nominal cell concentration and the fluorescence measurement for each dilution sample to determine at least one of linearity, range, accuracy, precision, limit of detection (LOD), and lowest limit of quantification (LLOQ) for the instrument and staining method. Determining the LLOQ may comprise, for example, identifying the concentration of target cells associated with a predetermined criterion for precision and a predetermined criterion for accuracy. Negatively staining the target cells in the reference sample first portion in (a) may comprise introducing to the reference sample first portion (i) a fluorescent dead cell exclusion dye, (ii) a non-specific antibody conjugated to a fluorochrome, for example as an isotype control, and (iii) a specific antibody capable of specifically binding a target marker (antigen) on the target cell, unconjugated to the fluorochrome. The non-specific antibody that is conjugated to the fluorochrome for negative staining, may comprise an antibody lacking the capability of specifically binding to a cell antigen, such as for example, IgD, IgG, IgA, IgM or IgE. The negatively stained first portion may be prepared in the absence of a cell-depletion step. Positively staining the target cells in the reference sample second portion in (b) may comprise introducing to the reference sample second portion (i) the fluorescent dead cell exclusion dye (the same dye as used for negatively staining), and (ii) and same the specific antibody used for the negative staining, but not unconjugated to the fluorochrome, rather conjugated to the same fluorochrome used for the negative staining (in which the fluorochrome is conjugated to the non-specific antibody). In any of the methods, the fluorescent dead cell exclusion dye used for staining may be selected from a nucleic acid binding dye, propidium iodide, DAPI, DRAQ7, 7-AAD, TO-PRO-3, and an amine-reactive dye. The fluorochrome used may be selected from known fluorochromes, for example but not limited to Allophycocyanin (APC), APC C750, APC AF700, brilliant violet (BV)421, BV510, hilite 7 (H7) BV605, BV650, PE CF594, Fluorescein isothiocyanate (FITC), R-Phycoerythrin (PE or R-PE), PE-Cy7 (PE coupled to cyanine dye Cy7), APC-Cy7 (APC coupled to cyanine dye Cy7), APC-H7 (APC coupled to the Cy analogue Hilite 7 (H7)). The target cell marker may be selected from, for example, CD3, CD4 and CD8 as a T-cell marker, CD19 as a B-cell marker, CD235a as an erythrocyte marker, CD56 as a Natural Killer (NK) cell marker, CD14 as a monocyte marker, and CD66b as a granulocyte marker. In one aspect, the target cell population is a T cell population, and the target cell marker is CD3.

The method may further comprise multiplexing by negatively staining and positively staining for two or more different target markers of the target cells, or potentially for staining for two or more different target markers of two or more target cells. The negatively staining may further comprise introducing to the reference sample first portion two or more specific antibodies, each specific antibody capable of specifically binding one of the two or more target markers and unconjugated to the fluorochrome. The positively staining may further comprise introducing to the reference sample second portion the two or more specific antibodies, each conjugated to the fluorochrome.

The calculating in (f) may be performed by a processor coupled with the instrument or flow cytometer. The test sample may comprise the target cell present in a concentration of no more than 2%, 1%, 0.5%, 0.2%, 0.1%, 0.05%, or 0.02%. The dilution series may comprise a dilution sample having a highest concentration of the target cells, wherein the highest concentration is 2%, 1%, 0.5%, 0.2%, 0.1%, 0.05%, or 0.02%. In any of the methods, the series of dilution samples may comprise at least two, three, four, five, six, seven, eight, nine or ten dilution samples.

In another aspect, the present disclosure contemplates a non-transitory computer-readable medium comprising instructions for a computer processor for performing the comparison in step (f) of any of the disclosed methods, or for performing any of the statistical calculations to determine any one or more of linearity, range, accuracy, precision, limit of detection (LOD) and lowest limit of quantification (LLOQ) for the instrument and staining method.

In still another aspect, the present disclosure provides a system for validating fluorescence measurements in a test sample made by a fluorescence-based instrument, the system comprising: the fluorescence-based instrument; and a computer coupled with the fluorescence-based instrument and comprising a computer-readable medium comprising instructions for a computer processor as described above. In the system, the fluorescence-based instrument may be a flow cytometer.

In yet another aspect, the present disclosure describes a kit comprising reagents for staining cells in a reference sample for validating a fluorescence-based analytical method for analyzing target cells in a test sample, the kit comprising: (i) negative stain reagents comprising: (a) a fluorescent dead cell exclusion dye, (b) a non-specific antibody conjugated to a fluorochrome, and (c) an unconjugated, specific antibody capable of specifically binding a target marker (antigen) on the target cells; (ii) positive staining reagents comprising: (a) the fluorescent dead cell exclusion dye, and (b) the specific antibody conjugated to the fluorochrome; and instructions for (a) negatively staining a first portion of the reference sample, (b) positively staining a second portion of the reference sample, and (c) preparing a dilution series comprising a plurality of dilution samples each having a nominal concentration of the target cells varying systematically across the dilution series, wherein the nominal cell concentration of at least one dilution sample is greater than the concentration of target cells indicated by fluorescence measurement of the negatively stained first portion.

Other aspects and features of the disclosure are detailed below.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

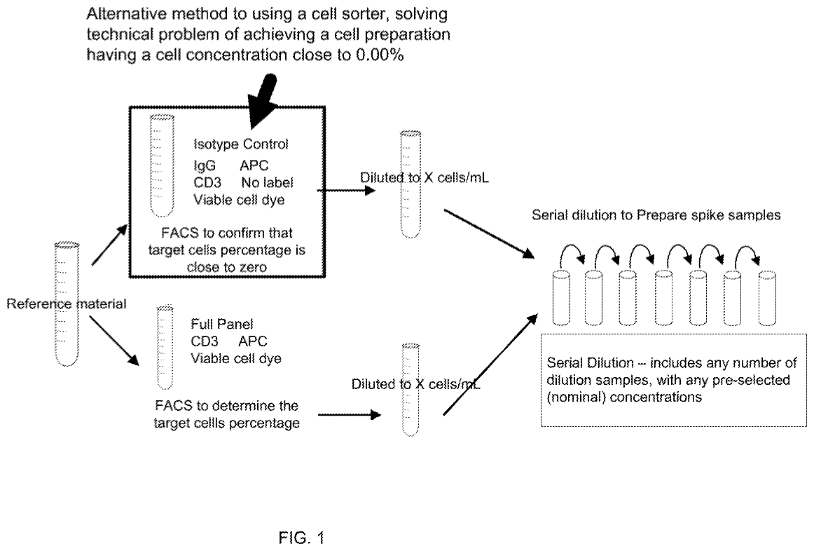

FIG. 1 provides a schematic view of a method according to the present disclosure.

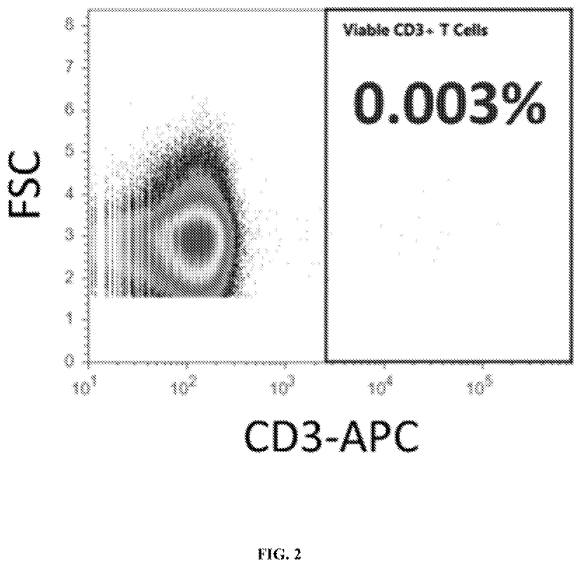

FIG. 2 is a FACS plot showing typical results obtained with the negatively stained sample. Events shown were gated for viable cells based on forward and side scatters, and PI (for dead cell exclusion).

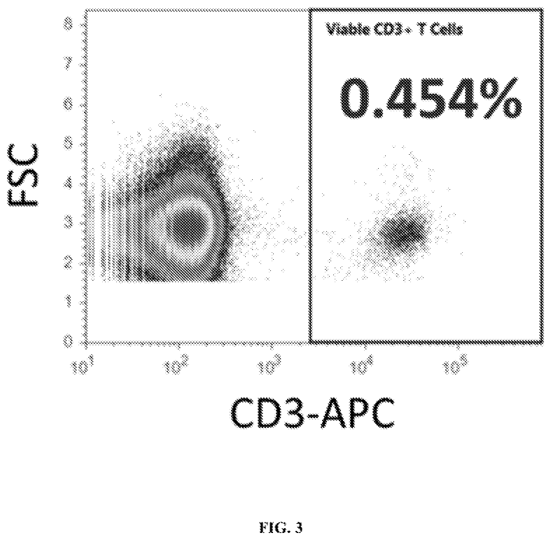

FIG. 3 is a FACS plot showing a typical results obtained with the positively stained sample. Events shown were gated for viable cells based on forward and side scatters, and PI (for dead cell exclusion).

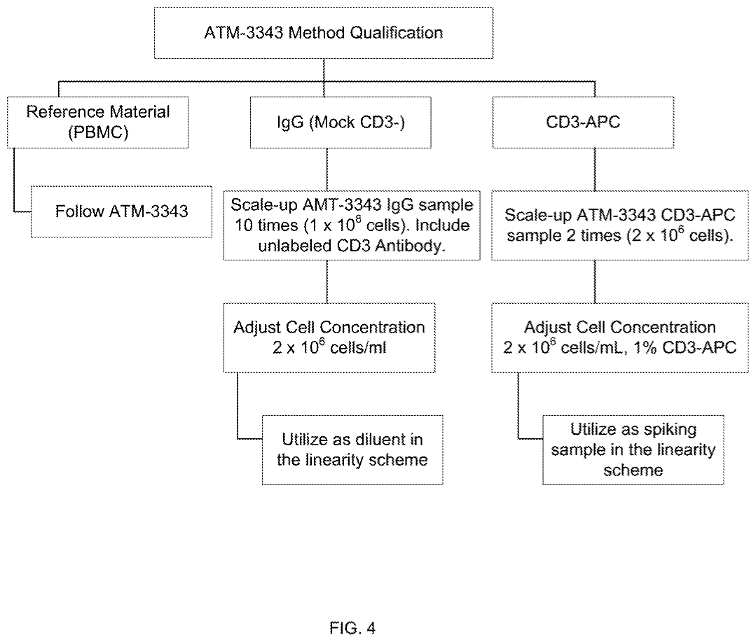

FIG. 4 illustrates the procedural flow for preparation of the linearity samples.

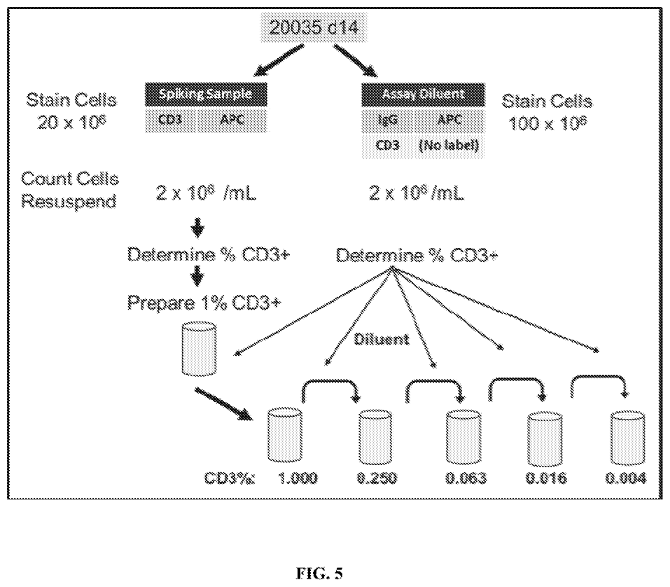

FIG. 5 illustrates the procedural flow for preparation of the dilution series.

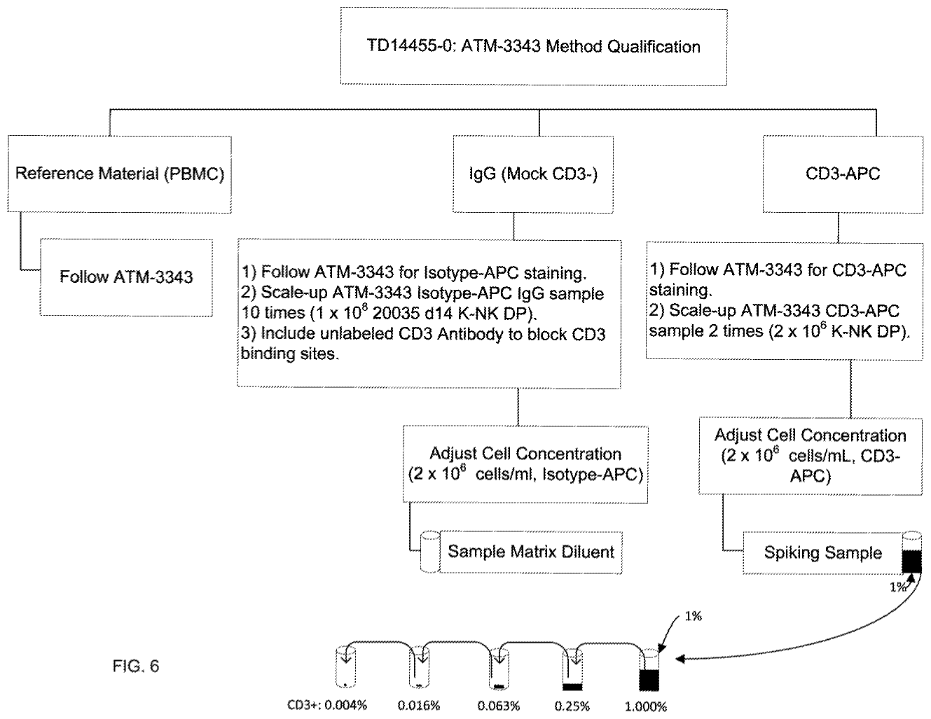

FIG. 6 illustrates the execution of method qualification by two operators at three different occasions specified in Example 2.

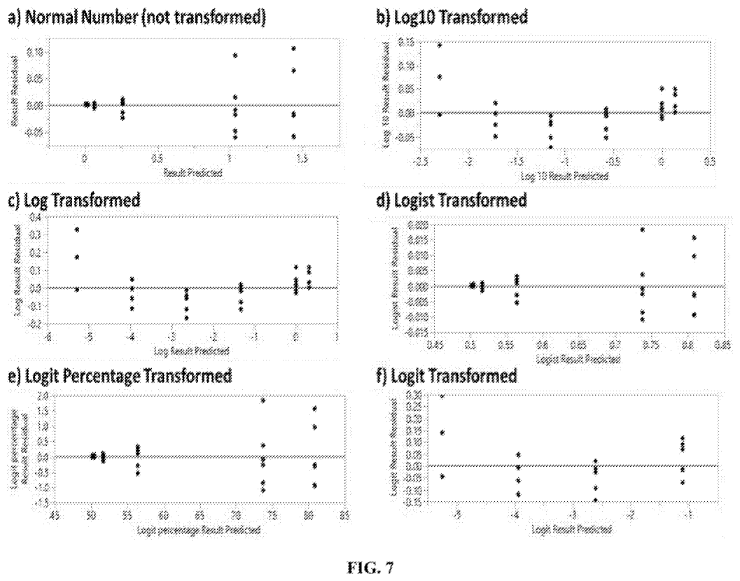

FIG. 7 is a residual plot of untransformed Nominal CD3% and Estimated CD3% (Result) of a) untransformed data and b)-f) different log transformed data.

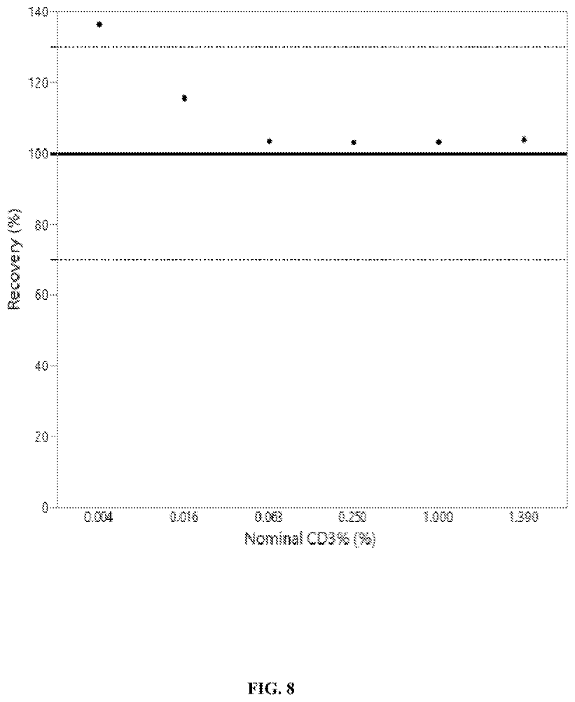

FIG. 8 depicts the percentage (%) of Estimated CD3% compared to Nominal CD3%. Dotted lines indicate 100 ±30% recovery.

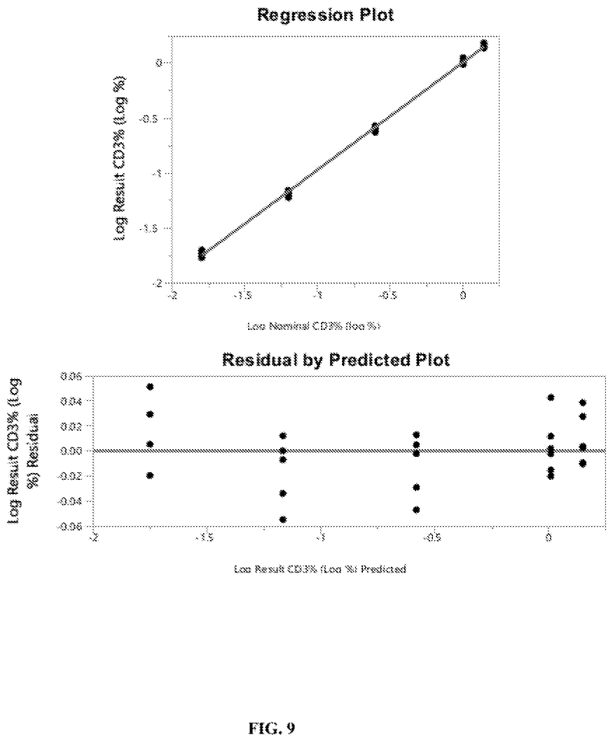

FIG. 9 illustrates the regression analysis of estimated linearity samples.

### DETAILED DESCRIPTION

Analytical method validation is critical for assessing the credibility and reproducibility of fluorescence-based analytical measurements. For such measurements to be valuable, they must be reproducible. For example, fluorescence-activated cell sorting (FACS) is a specialized type of fluorescence-based analysis and is a powerful method for sorting a mixture of biological cells into separate populations based upon fluorescent signals of each cell. In contrast to flow cytometry performed to merely count and sort cells, FACS provides both qualitative and quantitative analysis using flow cytometry data. It is important that fluorescence-based analytical measurements such those made using FACS be properly validated because of variation in the quality and performance of the antibodies used in such analytical techniques.

One validation approach relies on the preparation of a dilution series of multiple dilution or spiked samples each having a known or nominal concentration of the target cell or marker. For example, serial dilutions are performed by spiking known quantities of a target cell to generate multiple spiked samples with known (nominal) cell concentrations ranging systematically from a higher concentration, e.g. 50% to a lower concentration, e.g., 1% of the cells. The spiked samples are run through the cytometer and fluorescence measurements obtained for each spiked sample. Spiked samples are compared to the measured values from the cytometer. Accuracy of the instrument and staining method is determined by statistical calculations, e.g., by calculating the correlation of the nominal and measured values, determining slope of the relationship, determining R-sq, and so on. The FACS Limit of Detection (LOD) and/or Limit of Quantificaton (LOQ) can be calculated as is known, from staining and acquiring replicate unspiked sample(s) and running through the cytometer to obtain measures of positive events. Validated assays can then be used assays to detect and quantify the concentration of cells a sample.

To prepare a series of spiked samples accurately, at least two samples are required: one or more samples each having a known (nominal) concentration of the target cell or marker, and a sample that contains approximately none of the target cell or marker (concentration of about 0). More than one spiked sample is typically used. For example, validating an instrument and staining method using five (5) levels involves preparing a series of spiked samples containing five different, nominal concentrations of the target cell or marker (e.g., 2%, 4%, 6%, 8%, and 10%), plus one sample containing 0%. To prepare the series of spiked samples, a reference material containing a known, relatively higher concentration of the target cell or marker of interest is successively and systematically diluted to prepare the series of spiked samples having lower concentrations. However, in many instances, preparing spiked samples is not easy because no off-the-shelf starting reference material is available. For example, when the analyte of interest is a cell, such as a CD3⁺ cell (a T-cell), no such standard reference material exists. In other words, one cannot simply purchase a standard reference material having a known concentration, such as 10% CD3⁺ cell or 0% that can be diluted to prepare spiked samples having the lower concentrations of 8, 6, 4 and 2%. In theory, commercially available cell sorters could be used to prepare a series of spiked samples with successively lower concentrations. However, such an approach is expensive and time-consuming when the estimated concentrations of the target cell or marker are low, e.g. about 0.1% or lower, as low as about 0.01%.

The present disclosure solves the technical problem of providing a negative reference and spiked samples for a situation when no standard reference material exists, and by providing for the preparation of spiked samples lower than 0.1%, is useful for validation when the target cell or marker is present in a concentration lower than 0.1%. For example, depending on the instrument and staining being used, the LLOQ can be lower than 0.01%. Accordingly, the present disclosure relates to methods and systems for validating measurements by a fluorescence-based analytical instrument, such as a flow cytometer, of a stained target cell population in a test sample.

### I. Fluorescence-Based Analytical Instruments

A fluorescence-based analytical instrument encompasses any analytical instrument capable of measuring a fluorescence signal as an indicator of the presence of a target analyte or target cell type in a sample. Depending on the instrumentation and methods used, the instrument may be capable of quantifying the amount of target analyte or cell type in test a sample based on the fluorescence signal strength. One example of a fluorescence-based analytical instrument is a flow cytometer. FACS (fluorescence activated cell sorting) is a different but related methodology based upon flow cytometry. Flow cytometry is a method using a flow cytometer to examine and determine the expression of molecules both internal and external to a cell, and define and characterize distinct single cell types. Flow cytometry can also be used to determine other parameters such as cell size, volume, and purity of an isolated cell sample. FACS is a flow cytometry technique utilizing specific antibodies labeled with fluorochromes to obtain expression data and sort cell samples by a number of variables.

FACS can be performed using any one of a number of flow cytometer types, including traditional flow cytometers, Acoustic Focusing Cytometers, Cell Sorters, or Imaging Flow cytometers. The most common, traditional cytometers use sheath fluid for focusing the sample stream, and common lasers such as 488 nm (blue), 405 nm (violet), 532 nm (green), 552 nm (green), 561 nm (green-yellow), 640 nm (red) and 355 nm (ultraviolet). In Acoustic Focusing Cytometers, ultrasonic waves are used to focus the cells for analysis while preventing the sample from clogging and allowing higher sample inputs. Cell sorters are a type of traditional flow cytometer allowing the user to collect samples after processing. Cells that are positive for the desired parameter can be separated from those that are negative for the parameters. Imaging cytometers are traditional cytometers combined with fluorescence microscopy and all for rapid analysis of a sample for morphology and multi-parameter fluorescence at both a single cell and population level. Flow cytometers are routinely coupled with a computer having a processor that controls function of the instrument. The methods and systems described herein may be applied to use of any flow cytometer described herein.

### II. Methods

The methods described herein address the technical problem of providing a negative reference and low-level spiked samples in a situation when no standard reference material exists, thus allowing for reliable validation of fluorescence-based measurements, e.g., by a flow cytometer (FACS), in situations where a target cell or marker is believed present in a sample at a concentration lower than 0.1%, or even lower.

FIG. 1 is a schematic illustration of the method. Positively and negatively stained reference samples are prepared using an antibody capable of specifically binding a target cell marker (specific antibody), and an “isotype” (non-specific antibody), and a fluorochrome conjugated to the specific antibody or to the non-specific antibody, depending on whether the negative or positive staining is being performed. More specifically, a reference sample is obtained and divided into two portions. A first portion of the reference sample is negatively stained with (i) a fluorescent dead cell exclusion dye, (ii) the non-specific antibody conjugated to the fluorochrome, for example as an isotype control, and (iii) the specific antibody capable of specifically binding the target marker (antigen) on the target cell. For the negative staining, the specific antibody is unconjugated to the fluorochrome. The non-specific antibody conjugated to the fluorochrome for negative staining is an antibody capable of binding to a cell that does not have an epitope specifically for that antibody, i.e., binds to the cell but not by specific binding as in binding of an antibody to a specific epitope on the cell, and thus the non-specific antibody also has a high dissociation constant (Kd) for the marker, as described in more detail below. Any one of the non-specific immunoglobulins IgD, IgG, IgA, IgM or IgE can be used for the non-specific antibody.

The second portion of the reference sample is positively stained with the fluorescent dead cell exclusion dye (the same dye used for the negative staining step), and the same, specific antibody used for the negative staining, but not unconjugated, rather conjugated to the same fluorochrome used for the negative staining (in which the fluorochrome is conjugated to the non-specific antibody).

The target cell marker may be any cell marker such as any known marker of a T-cell, B-cell, NK cell, erythrocyte, granulocyte or monocyte. T-cell markers for example include but are not limited to CD3, CD4, CD8, CD69, CD71 and CD25. B-cell markers include, but are not limited to IL-6, CD19, CD25, CD30, CD27, CD38, CD78, CD138, and CD319. In one aspect, for example, the target marker may be selected from CD3 as a T-cell marker, CD19 as a B-cell marker, CD235a as an erythrocyte marker, CD56 as a Natural Killer (NK) cell marker, CD14 as a monocyte marker, and CD66b as a granulocyte marker. It will be understood that validation with respect to more than one marker of a cell population can readily be performed by applying the method to multiple samples from same reference material, using an appropriate specific antibody in each instance. By way of non-limiting example, to validate two different staining protocols for two different markers for a T-cell population, the method may be performed a first time using an antibody that specifically binds CD3, and performed a second time using an antibody that binds specifically to CD4.

It will be understood that the selection of the target marker drives the selection of the specific for the staining. Specific binding of the antibody to the target marker refers to the ability of the antibody to recognize epitope(s) of the marker such that the selected antibody exhibits high binding affinity to the marker, and preferably low binding affinity for other cross-reacting species. Generally, the binding affinity of a molecule is described using the dissociation (or equilibrium) constant (Kd). The lower the dissociation constant, the greater the binding affinity or degree of binding antibody A has for analyte (marker) B. Thus, a specific antibody for a marker exhibits a low dissociation constant (Kd) for the marker. Methods of determining the Kd for a given antibody are very well described in the literature as further noted below. Antibodies that generate poor signal at low concentrations will have high variability (noise) and thus will be difficult to discriminate from higher measured cross-reactivity. A “specific antibody” as used herein refers to an antibody having a Kd for the target marker of no greater than 10 nM or lower, no greater than 7 nM or lower, no greater than 5 nM or lower, no greater than 1 nM or lower, no greater than 0.5 nM or lower, no greater than 0.15 nM, or no greater than 0.1 nM, or lower than 0.1 nM. By way of non-limiting example, a specific antibody may be selected which has specific binding to the target marker with a Kd of 5 nM or lower, such as 2 nM or lower, preferably 1 nM or lower, and more preferably 0.7 nM or lower. The Kd value describing the binding affinity of an antibody considered specific for the target marker is provided by commercial suppliers of commercially available antibodies, or can be determined by well known methods including without limitation: fluorescence titration, competition ELISA, calorimetric methods, such as isothermal titration calorimetry (ITC), flow cytometric titration analysis (FACS titration) and surface plasmon resonance (BlAcore). Such methods are well described in the literature. (See, e.g., Goodrich & Kugel, 2007, Binding and Kinetics for Molecular Biologists, Cold Spring Harbour Laboratory Press, Cold Spring Harbour, N.Y.; De Jong, L. A. A. et al., J. Chromatogr. B 829(1-2):1-25 (2005); Heinrich, L. et al. J. Immunol. Methods 352(1- 2):13-22 (2010); Williams, M. A. & Daviter, T. (Eds.) 2013, Protein-Ligand Interactions, Methods and Applications, Springer, New York, N.Y.).

Accordingly, a specific antibody in the present disclosure is any antibody having a Kd for no greater than 1 nM, no greater than 0.5 nM, no greater than 0.15 nM, or no greater than 0.1 nM for any of the target cell markers, including but not limited to any of the cell markers described herein: CD3, CD4, CD8, CD69, CD71 and CD25, IL-6, CD19, CD25, CD30, CD27, CD38, CD78, CD138, and CD319. CD235a, CD56, CD14 and CD66b. Specific antibodies may be polyclonal, monoclonal, chimeric, humanized, or fully human antibodies. They can be single chain or multi-chain antibodies. While suitable specific antibodies can and commonly are purchased from commercial suppliers, monoclonal antibodies may also be produced from hybridomas, or in host cells containing vectors comprising nucleic acid sequences encoding the antibodies, which the cells then express. As such, specific antibodies as described herein can be made by transforming a host cell with at least one nucleic acid molecule encoding the specific antibody; expressing the nucleic acid molecule in the host cell; and isolating the specific antibody, all using materials and techniques well known in the art. Commercial suppliers for specific antibodies for a wide range of cell markers include but are not limited to: BD Biosciences, Takara Bio USA (e.g., offers monoclonal antiCD3 (OKT3))); BioX Cell (Lebanon, N.H.; e.g., offers anti-human CD19 monoclonal antibody); Thermo-Fisher Scientific Inc.; BioLegend Inc.; Bio-Rad Antibodies, among others.

The fluorescent dead cell exclusion dye used in both the negative staining and positive staining steps may be any dye that allows discrimination of viable cells from dead or dying cells, and are also known as viability dyes. For example, viability dyes include those that are not membrane permeable, do not pass cell membranes. For example, nucleic acid binding dyes that do not penetrate cell membranes will selectively stain the accessible nucleic acid (e.g., DNA) of dead and dying cells, and not stain the inaccessible nucleic acid inside viable cells with intact membranes. Alternatively, the dead cell exclusion or viability dye can be a protein binding dye (amine-reactive dye) rather than a nucleic acid binding dye. Protein binding dyes bind to both live and dead cells, but dead and dying cells with disrupted membranes are stained more intensely by the dye which has access to more intracellular protein, and thus the dead and dying cells exhibit higher fluorescence. Thus, using either a nucleic acid binding dye, or an amine-reactive dye, dead cells can be excluded by gating on the less intensely stained population which comprises live cells. Dead cell exclusion dyes include numerous and readily commercially available nucleic acid binding dyes and amine-reactive dyes, such as but not limited to propidium iodide, DAPI, DRAQ7, 7-AAD, TO-PRO-3, live/dead fixable dyes, the eFluor fixable dyes, the Horizon dyes, the Biolegend Zombie dyes and the Ghost dyes. The amine-reactive dye includes M1420MP, M1410, D6105, P130, P6114, A10168, M10165, D10161, D374, D126, D1421, B30250, H185, H1428, H1193, P30253, A30000, A30100, C10164, P10163, S6110, D2184, D2183, D3834, and others.

The fluorochrome may be any which has a characteristic, visible emission spectrum, such as but not limited to any one of many known fluorochromes currently commercially available, such as Allophycocyanin (APC), APC C750, APC AF700, brilliant violet (BV)421, BV510, hilite 7 (H7) BV605, BV650, PE CF594, Fluorescein isothiocyanate (FITC), R-Phycoerythrin (PE or R-PE), PE-Cy7 (PE coupled to cyanine dye Cy7), APC-Cy7 (APC coupled to cyanine dye Cy7), APC-H7 (APC coupled to the Cy analogue Hilite 7 (H7)).

Target marker binding sites in the negatively stained sample (isotype stained sample) are blocked using the unconjugated version of the specific antibody, i.e., the specific antibody to which no fluorochrome is conjugated or covalently attached. The positively stained sample is then used to prepare at least two or usually more than two spiked samples, by spiking to the target nominal cell concentrations using the negatively stained cells. The series of spiked samples are then analyzed in a flow cytometer to obtain a measured concentration from the instrument, for each spiked sample. The data (the nominal and the measured concentration for each spiked sample) are plotted and statistically analyzed for correlation and to calculate validation parameters such as Linearity, Accuracy, Precision, LOD and/or LLOQ. It will be understood that in any of the methods, analyzing the comparing the nominal cell and measured cell concertation data to quantify the performance of the staining method in the instrument, may be performed by a computer processor coupled with the instrument, such as a processor configured with instructions to performing one or more standard statistical tests of the differences between each nominal cell concentration and fluorescence measurement for each dilution sample, to determine at least one of linearity, range, accuracy, precision, LOD and LLOQ for the instrument and staining method. Determining the LLOQ may comprise, for example, identifying the concentration of target cells associated with a predetermined criterion for precision and a predetermined criterion for accuracy.

Example 1 below illustrates the methods as used for determining viable T-cell concentration in a cell product sample, using the propidium iodide as the dead cell exclusion dye, an anti-CD3 antibody, IgG as the nonspecific antibody and allophycocyanin as the fluorochrome. It should however be understood that any one of the dead cell exclusion dye, cell marker (CD3) and specific antibody (anti-CD3) pairing, nonspecific antibody (IgG), and/or fluorochrome (APC) in the example may be substituted as described herein. Following negative and positive staining, the positively stained sample is diluted to the highest desired spike level (highest nominal concentration of the target cells) planned, using the negatively stained cells, and then serial dilutions are prepared to generate dilution samples with systematically varying nominal cell concentrations. A dilution series may comprise any multiple number. In theory even two dilution samples could be used, however in practice, at least three dilution samples are prepared, each varying in nominal concentration, preferably systematically to facilitate analysis. A dilution series may include three, four, five, six, seven, eight, nine, ten, eleven, twelve or more dilution (spiked) samples. By way of non-limiting example, the nominal concentration of each dilution (spiked) sample may vary from 0.1% down to 0.003%, but it will be understood that the selection of nominal values will vary with a range of factors such as the expected concentration of the target cell or marker in the sample, the number of spiked samples being used, instrumentation, staining protocol being used, etc.

It will further be understood that the present methods and systems address the problem of providing a negative sample for a test sample that may comprise the target cell present in a concentration of no more than 2%, 1.5%, 1%, 0.8%, 0.7%, 0.6%, 0.5%, 0.4%, 0.3%, 0.25%, 0.2%, 0.15%, 0.1%, 0.05%, 0.02%, or no more than 0.01%. Comparably, the dilution series may comprise a dilution sample having a highest concentration of the target cells, wherein the highest concentration is 2%, 1.5%, 1%, 0.8%, 0.7%, 0.6%, 0.5%, 0.4%, 0.3%, 0.25%, 0.2%, 0.15%, 0.1%, 0.05%, 0.02%, or 0.01%. For example, the methods provided herein may be used to determine a target cell or marker considered residue or impurity in a sample. For example, in a composition of natural killer cells, CD3+ cells or T-cells may be considered a residue or impurity. The present method may allow a determination of residual or impurity to a very low level, such as less than 2%, 1.5%, 1%, 0.8%, 0.7%, 0.6%, 0.5%, 0.4%, 0.3%, 0.25%, 0.2%, 0.15%, 0.1%, 0.05%, 0.02%, or 0.01%.

### III. Systems

The present disclosure also provides systems and devices comprising any one or more fluorescence-based instruments, and related system components for implementing the disclosed methods and their various aspects and features. The systems may include for example one or more fluorescence-based instruments, one or more computer processor(s) coupled to the instrument(s), and optionally include one or more of any cell samples, dyes, fluorochromes, cell markers, antibodies, or any combination thereof. For example, the present disclosure contemplates a non-transitory computer-readable medium comprising processor executable instructions for the processor to perform any of the data analysis or statistical calculations on the data generated by the instrument as described herein. A system for validating fluorescence measurements made by a fluorescence-based instrument may include the fluorescence-based instrument, the computer coupled thereto, and the computer readable medium containing the processor executable instructions. As detailed herein further above, the instrument may be a flow cytometer, which may be used for performing FACS.

### IV. Kits

The present disclosure also contemplates kits and devices comprising any one or more cell samples, dyes, fluorochromes, cell markers, specific an/or non-specific antibodies, buffers, diluents, or any combination thereof for performing any of the validation methods described herein. A kit may comprise, for example, any one or more of the reagents in amounts needed for negatively and/or positively staining cells in a reference sample for validating a fluorescence-based analytical method for analyzing target cells in a test sample. The kit may comprise the components needed for the negative staining, including any one or more of a fluorescent dead cell exclusion dye, a non-specific antibody conjugated to a fluorochrome, and an unconjugated, specific antibody as described herein, which is capable of specifically binding a target marker (antigen) on the target cells. The kit may comprise the components needed for the positive staining including any one or more of the fluorescent dead cell exclusion dye, and the specific antibody conjugated to a fluorochrome as described herein.

It will be understood that the specific antibody for both the negative staining and positive staining will be the same, given a selected target cell marker. Similarly, the fluorochrome in the kit for both the negative staining and the positive staining is the same. The kit may include written instructions in hard copy or electronic format, for the negative staining and positive staining of the reference sample, and for preparing the dilution series as described herein above. Instructions may additionally include any one or more instructions for: using the kit to perform any of the validation methods described herein; a preparing a reference sample; and/or reading, analyzing, and interpreting the results of the validation method. The kit may usefully include one or more containers for preparing and/or storing any of the reagents, and components for labeling them.

### DEFINITIONS

As various changes could be made in the above-described apparatus, kits and methods without departing from the scope of the invention, it is intended that all matter contained in the above description and in the examples given below, shall be interpreted as illustrative and not in a limiting sense.

Unless defined otherwise, all technical and scientific terms used herein have the meaning commonly understood by a person skilled in the art to which this invention belongs. The following references provide one of skill with a general definition of many of the terms used in this invention: The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “comprising” means “including, but not necessarily limited to”; it specifically indicates open-ended inclusion or membership in a so-described combination, group, series and the like. The terms “comprising” and “including” as used herein are inclusive and/or open-ended and do not exclude additional, unrecited elements or method processes. The term “consisting essentially of” is more limiting than “comprising” but not as restrictive as “consisting of.” Specifically, the term “consisting essentially of” limits membership to the specified materials or steps and those that do not materially affect the essential characteristics of the claimed invention.

### EXAMPLES

Any publications discussed herein is provided solely for its disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Generation of Spiked Samples for Validation of Flow Cytometry Analysis

In this example, spiked samples were generated for validation of flow cytometry analysis of a “rare events target”, for determination of LLOQ (lowest level of quantification) for a residue or impurity present in a sample.
I. Method

FIG. 1 is a schematic illustration of the method. Positively and negatively stained cell samples were prepared using the “fluorochrome conjugated target marker” (specific antibody) and “isotype,” (non-specific antibody) respectively. Target marker binding sites in the negatively stained sample (isotype stained sample) were blocked by using the unconjugated version of the specific antibody, i.e., antibody to which no fluorochrome was covalently attached. The positively stained sample was then spiked to the target nominal cell percentages by using the negatively stained cells. The spiked samples were analyzed in a flow cytometer to obtain measured percentages. The data (the nominal and measured percentages of the spiked samples) were then used for statistical analysis to calculate validation parameters such as Linearity, Accuracy, Precision and LLOQ.
II. Sample Preparations

This example describes validating the viable T cell percentage in a cellular product using the following staining panel:

| Dye | Target cell population |
|---|---|
| PI | Dead Cells |
| Anti CD3-APC | T-cells |

PI = Propidium Iodide and APC = Allophycocyanin
Viable T-cells are identified as PI− CD3+.

a) Preparation of Negatively Stained Sample

A cellular test sample was divided into two portions. One portion was stained according to the manufacturer protocols using the designed staining panel: combination of fluorochrome-conjugated antibody and dead cell exclusion dye necessary to gate the viable cell population of interest. For the target cell population, an isotype control was used in the staining mixture. In this example, this staining mixture used was:

Dye

PI
IgG Isotype-APC
PI = Propidium Iodide, IgG = immunoglobin G and APC = Allophycocyanin

After the staining, unconjugated antibody of the target marker was added to the final cell solution. In this example, the unconjugated antibody was purified anti-CD3 antibody. As shown in FIG. 2, a FACS plot showed the result obtained with the negatively stained sample. Events shown here were gated for viable cells based on forward and side scatters, and PI (for dead cell exclusion).
b) Preparation of Positively Stained Sample

From the cellular test sample in step 1, the other portion of cells according to the manufacturer protocol was stained using the designed staining panel:

Dye

PI
Anti CD3-APC
Note:
PI = Propidium Iodide and APC = Allophycocyanin

As shown in FIG. 3, FACS plot showed the result obtained with the positively stained sample. Events shown here were gated for viable cells based on forward and side scatters, and PI (for dead cell exclusion).
c) Preparation of Low-Level T-cell Spiked Samples

The positively stained sample was diluted to the highest spike level (nominal concentration of the target cells) planned, using the negatively stained cells from step 1), and then serial dilutions were prepared to generate dilution samples with systematically varying nominal T-cell percentages.
III. Analysis of Samples

Spiked samples were prepared as described above, and used to obtain fluorescence measurements from a flow cytometer, each measurement corresponding to a nominal concentration of CD3+ cells in a spiked sample. Each nominal concentration and corresponding fluorescence measurement from the flow cytometer was subjected to routine statistical routine analyses as described elsewhere herein, to assess the accuracy and reliability of the measurements.

This example describes the novel preparation of negatively stained cells using the fluorochrome conjugated isotype and the unconjugated antibody. Normally, target cell population depleted cells, prepared by magnetic beads or by cell sorter, are used to prepare the negative population for spiked samples preparation. However, these depletion procedures rarely deplete up to 99.9% of the target population. As a consequence, the negative population would contribute to a background of at least 0.1%, so that preparation of spiked samples lower than 0.1% is impossible. To validate a fluorescence-based analytical method such as FACS to an LLOQ lower than 0.1% is challenging. This example demonstrates effective preparation of a negative population having a target cell concertation lower than 0.1%, and allowed the preparation of spiked samples lower than 0.1% for validation. Depending on the instrumentation and staining panel used, the present methods can provide an LLOQ lower than 0.01%.

### Example 2. Qualification Protocol for a T Cell Quantification Method of K-NK Drug Product

This method qualification protocol provides the procedure for qualification of a T cell quantification method (Protocol reference: ATM-3343), for quantification of CD3+ expressing cells in a stimulated, expanded Natural Killer cell composition, (K-NK Drug Product (DP)), such as those disclosed in PCT publication WO2018160673A1, the disclosure of which is herein incorporated by reference in its entirety. The K-NK DP was a CD3+ depleted donor lymphocyte preparation from which NK cells are expanded to high numbers and density in vitro using 21-41bbl plasma membrane (PM21) particles, such as those described in WO2018160673A1. A primary impurity in K-NK DP was residual CD3+ expressing cells, which may elicit graft versus host disease if administered to patients over a clinically relevant limit. To detect and provide quantification of low concentration residual CD3+ expressing cells in K-NK DP, the FACS based method described herein was developed to detect and/or quantify viable CD3+ cell content exclusively. The definitions provided in Table 1 and Table 2 are used throughout the following examples.

TABLE 1
DEFINITIONS USED IN EXAMPLES

| Term | Definition |
|---|---|
| Accuracy/Trueness | The accuracy of an analytical procedure expresses the |
| | closeness of agreement between the value which is accepted |
| | either as a conventional true value or an accepted reference |
| | value and the value found. This is also termed trueness. For |
| | biological assays for which no official reference standard exists, |
| | this parameter can be determined as relative accuracy. |
| Intermediate | Intermediate Precision expresses within-laboratories |
| Precision | variations: different days, different analysts, different |
| | equipment, etc. |
| Linearity | The Linearity of an analytical procedure is its ability (within a |
| | given range) to obtain test results which are directly |
| | proportional to the concentration (amount) of analyte in the |
| | sample. |
| Precision | The Precision of an analytical procedure expresses the |
| | closeness of an agreement (degree of scatter) between a |
| | series of measurements obtained from multiple sampling of the |
| | same homogenous sample under the prescribed conditions. |
| | Precision may be considered at 3 levels: Intermediate |
| | Precision and Reproducibility. |
| Quantitation | The lowest amount of analyte in a sample that can be |
| Limit (LOQ) | quantitatively determined with suitable accuracy and |
| | precision. |
| Range | The range of an analytical procedure is the interval between |
| | the upper and lower concentration of analyte in the sample for |
| | which it has been demonstrated that the analytical procedure |
| | has a suitable level of precision, (relative) accuracy and |
| | (dilutional) linearity. |
| Recovery (%) | =(% estimated in test sample ÷ % estimated in reference |
| | sample) × 100% |

TABLE 2
ABBREVIATIONS and ACRONYMS USED IN EXAMPLES

| Acronym/Abb | Definition |
|---|---|
| % RSD | Percent Relative Standard Deviation = (SD/Mean) × 100 |
| APC | Allophycocyanin |
| ATM | Analytic Test Method |
| CD3 | T-cell marker protein complex |
| CSB | Cell Staining Buffer |
| CV | Coefficient of Variation. Expressed as % CV (ratio |
| | of the standard) |
| DGS | Density Gradient Centrifugation |
| DP | Drug Product |
| FIO | For Information Only |
| ICH | International Conference on Harmonization |
| MFI | Mean Fluorescence Intensity |
| MNC | Mononuclear Cells |
| MQ | Method Qualification |
| PBMC | Peripheral Blood Mononuclear Cells |
| PI | Propidium Iodide |
| PM21 | Plasma Membrane 21-41bbl particles |
| particles | |
| PPE | Personal Protective Equipment |
| R2 | Coefficient of Determination |
| RM | Reference Material. A PBMC population prepared |
| | by Ficoll-mediated |
| RT | Room Temperature |
| SIP | Sample Injection Port |
| TS | Test Sample |
| MFI | Mean Fluorescence Intensity |
| CV | Coefficient of Variation. deviation to the mean × 100) |
| DGS | Density Gradient Centrifugation |
| DP | Drug Product |
| LOQ | Limit of Quantification |
| PI | Propidium Iodide |

A healthy donor PBMC (RM-PBMC 225398) was utilized as “Positive Control samples to verify CD3+ staining and to set the CD3+ gate for flow cytometry analysis. This PBMC was prepared from density gradient centrifugations. This positive control did not have associated acceptance criteria but was utilized for gate setting and verification of CD3-APC antibody addition and reactivity.

The test article utilized for this qualification was a cryopreserved NK cell sample “20035 d14”. The 20035 d14 samples were generated by further processing of NK cells expanded using the PM21 particle platform, and procured after day 7 and were representative of K-NK product. After procurement, these cells were cultured in the laboratory for seven days and cryopreserved at 5×107cells per vial on day 14. This material was cryo-formulated in CS10 and although it did not contain Plasmalyte or HSA, samples were washed in Cell Staining Buffer (CSB) and resuspended in CSB prior to staining via analytical procedure. Therefore, the matrix of all test samples was standardized going into the analytical procedure, regardless of the beginning formulation. Performance testing during method development determined a relative average viable CD3+ content of 1.4% (n=8) for the 20035 d14 sample.

Equipment used was Attune™ Classic Acoustic Focusing Flow Cytometer, and software used to collect and process data was Attune Cytometric Software v2.1 and JMP v14.

The analytical method and procedure were designed to detect and quantify CD3+ expressing cells at low concentrations. Briefly, high cell concentration sample was washed multiple times in FACS staining buffer, and then stained with CD3-APC antibody to detect CD3+ expressing cells and Propidium Iodide (PI) for dead cells. Then, sample was resuspended to a fixed concentration and acquired via the Attune Flow Cytometer. Flow cytometry data was processed using Attune Cytometric Software v2.1. In order to obtain resolution with precise and accurate quantification at 0.01% cell content, 1×106 viable MNCs were acquired. The flow cytometry gating strategy was defined as MNCs-*Singlets-*Viable MNCs-*Viable CD3+ per ATM-3343. The qualification was designed to demonstrate the method's suitability for detecting and quantifying the percent constituency of CD3+ expressing cells. Parameters reported include: % Viable CD3+ cells.

Test sample preparation: For linearity, accuracy/trueness, and precision analysis, cryopreserved test sample 20035 d14 was split into two preparations. One aliquot (1×10⁸ cells) was stained with IgG-APC and unlabeled CD3 antibody to generate a mock CD3-sample and the second aliquot (2×107 cells) was stained with CD3-APC. The Isotype stained (IgG-APC) sample was served as the diluent and matrix sample for spiking with the fully stained sample to assess linearity and relative accuracy of the method. The unlabeled CD3 antibody was utilized to block CD3 binding sites due to any carry-over of CD3-APC from spiking sample. Prior to acquisition, the cell density was adjusted to achieve an event rate of ˜1000 events/sec as per ATM-3343-0. The concentration of the initial positively stained CD3+ spiking sample and the negatively stained IgG-APC-stained sample is utilized to generate the dilution scheme. The CD3-APC stained samples were split into two samples; 1) to determine % CD3+, and 2) to serve as spiking sample. Likewise, one aliquot (one tenth of preparation) of the IgG-APC stained sample were utilized to determine % CD3+ and rest of the sample were utilized as diluent in the linearity scheme.

Sample Preparation: Samples were prepared according to ATM-3343 and scaled up as described in Table 3, including staining of Positive Control, IgG-APC and CD3-APC samples. Since 1×108 cells were stained with IgG-APC, the reagents utilized were scaled up by 10 times. Additionally, molar equivalents of unlabeled CD3 antibody were added to block CD3 binding sites. The CD3-APC staining was performed on 2×107 cells, so the reagents utilized in the ATM-3343 were doubled. Additional back-up tubes were not utilized. Each serial dilution sample had the same cell concentration of 2×106/mL (minimum 2 mL volume). The procedural flow for preparation of both linearity samples and the dilution series is illustrated in FIG. 4 and FIG. 5, respectively. A 4-fold dilution series was performed to five levels and included a dilutional range 1.00% to 0.004%. Spike samples were prepared according to Table 4. For each occasion, one positive Control, one IgG-APC and one CD3-APC samples were prepared and acquired.

TABLE 3
Data Worksheet: Preparation of Linearity Sample Matrix

Step 1: Sample Preparation
Note: Samples are acquired according to ATM-3343-0.
WATM-3343-0
Supplemental Information to scale up IgG-APC, IgG-APC (Mock CD3-) and CD3-APC:
IgG-APC Acquisition:
Sample: 20035-d14, record “Total Viable Cells”, “Volume ((μL)”, “TruStain Fc Block reagent ((μL)”, and
“IgG-APC (μL)”.
Total Viable Cells:
IgG-APC Acquisition (Mock CD3- Sample):
Sample: 20035-d14, and record “Total Viable Cells”, “Volume ((μL)”, “TruStain Fc Block reagent ((μL)”,
and “IgG-APC (μL)”.
Unlabeled Purified Anti-Human CD3:
Manufacturer: Biolegend, Catalog Number: 344802, Lot:
CD3-APC Acquisition:
Sample: 20035-d14, record “Total Viable Cells”, “Volume ((μL)”, “TruStain Fc Block reagent ((μL)”, and
“CD3-APC (μL)”.
Step 2: Preparation of the Mock CD3- diluent and CD3-APC spiking samples
IgG-APC (Mock CD3- Sample)
Transcribe results:
1 × 10⁶ Viable Single MNC Acquired: record if “Assay” is valid or invalid
Transcribe % Viable Single CD3+ from CD3-APC FACS Acquisitions:
Record “Percent Viable Single CD3+” in “%”
Record “Cell Concentration” in “cells/mL”
Preparation:
Adjust Sample concentration to 2.00 × 10⁶ cells/mL in 10 mL Final Volume
$Sample Volume = \frac{2. e 6 cells / mL \times 10 mL}{Cell Concentration}$
Record “Sample Volume” in “mL”
Record “Cell Staining Buffer Volume to QS 10 mL” in “mL”
CD3-APC:
Transcribe results:
1 × 10⁶ Viable Single MNC Acquired: record if “Assay” is valid or invalid
Transcribe % Viable Single CD3+ from CD3-APC FACS Acquisitions:
Record “Percent Viable Single CD3+” in “%”
Record “Cell Concentration” in “/mL”.
Preparation:
Adjust Sample concentration to 2.00 × 10⁶ cells/mL in 2 mL Final Volume
$Sample Volume = \frac{2. e 6 cells / mL \times 2 mL}{Cell Concentration}$
Record “Sample Volume” in “mL”
Record “Cell Staining Buffer Volume to QS 2 mL” in “mL”
Step 3: Preparation of CD3+ linearity samples
Serial 4-fold dilutions using 1.000% CD3+ as initial spiking sample
Change tips in-between sample transfer
Dilution Level 1, record if “performed”
Prepare a CD3-APC Sample from Step 2 at 1.000% CD3+; Sample Concentration = 2 × 10⁶/mL;
Sample volume = 2 mL
Record “Percent Viable Single CD3+” in “%” (from Step 2)
$Sample Volume = \frac{1. \% CD 3 + \times 2 mL}{\% Viable Single CD 3+}$
Record “Sample Volume” in “mL”.
Record “IgG-APC (Mock CD3- Sample, from Step 2) diluent sample volume to QS 2mL” in “mL”:
Dilution Level 2, record if “performed”
Prepare a CD3-APC Sample from Dilution Level 1 at 0.250% CD3+; Sample Concentration = 2 ×
10⁶/mL; Sample volume = 2 mL
Spiking sample = 0.500 mL (1% CD3+, Dilution Level 1)
IgG-APC (Mock CD3- Sample, from Step 2) diluent sample = 1.500 mL
Dilution Level 3, record if “performed”
Prepare a CD3-APC Sample from Dilution Level 2 at 0.063% CD3+; Sample Concentration = 2 ×
10⁶/mL; Sample volume = 2 mL
Spiking sample = 0.500 mL (0.250% CD3+, Dilution Level 2)
IgG-APC (Mock CD3- Sample, from Step 2) diluent sample = 1.500 mL
Dilution Level 4, record if “performed”
Prepare a CD3-APC Sample from Dilution Level 3 at 0.016% CD3+; Sample Concentration = 2 ×
10⁶/mL; Sample volume = 2 mL
Spiking sample = 0.500 mL (0.063% CD3+, Dilution Level 3)
IgG-APC (Mock CD3- Sample, from Step 2) diluent sample = 1.500 mL
Dilution Level 5, record if “performed”
Prepare a CD3-APC Sample from Dilution Level 4 at 0.004% CD3+; Sample Concentration = 2 ×
10⁶/mL; Sample volume = 2 mL
Spiking sample = 0.500 mL (0.016% CD3+, Dilution level 4)
IgG-APC (Mock CD3- Sample, from Step 2) diluent sample = 1.500 mL

Dilution Series Preparation

| | | CD3-APC CD3+ Spiking | CD3-APC CD3− Spiking |
|---|---|---|---|
| Dilution | Dilution | Sample vol | Sample vol |
| Level | Level | (2 × 10⁶ Cells/mL) | (2 × 10⁶ Cells/mL) |
| 1 | 1.000% | Dependent on CD3+ conc | Dependent on CD3+ conc |
| 2 | 0.250% | 0.500 mL (1.000%) | 1.500 mL |
| 3 | 0.063% | 0.500 mL (0.250%) | 1.500 mL |
| 4 | 0.016% | 0.500 mL (0.063%) | 1.500 mL |
| 5 | 0.004% | 0.500 mL (0.016%) | 1.500 mL |

The data set for qualification parameters linearity, accuracy/trueness, precision, and specificity were generated from 3 occasions performed by 2 operators as shown in FIG. 6. One test sample per occasion were processed according to the dilution scheme above and the qualification were executed per the scheme illustrated in Table 5 below. The samples were processed according to ATM-3343 (serial dilution were equivalent to the test samples). Dilution Level 5 were acquired first, followed by Dilution Level 4 and sequentially to the first dilution level. One data point per level per occasion was generated. Three measurements were made per level per operator for a total of six data points per dilution level for this qualification exercise.

TABLE 5
Method Qualification Execution Scheme

| | Occasion 1 | Occasion 2 | Occasion 3 | |
|---|---|---|---|---|
| | 1 dilution | 1 dilution | 1 dilution | Results |
| Operator | series | series | series | per level |
| A | 1 measurement | 1 measurement | 1 measurement | 3 |
| | per level | per level | per level | |
| | 1 RM-PBMC | 1 RM-PBMC | 1 RM-PBMC | |
| | 1 Isotype | 1 Isotype | 1 Isotype | |
| B | 1 measurement | 1 measurement | 1 measurement | 3 |
| | per level | per level | per level | |
| | 1 RM-PBMC | 1 RM-PBMC | 1 RM-PBMC | |
| | 1 Isotype | 1 Isotype | 1 Isotype | |

Relative accuracy/trueness: Relative accuracy or trueness were assessed using the mean value of the test material against the corresponding nominal relative percent CD3+ per dilution level. Intermediate precision were determined for occasion and analyst per dilution level.

The range of the procedure was determined from the dilutional linearity exercise. The lower limit of quantitation was determined from the linearity exercise as the lowest dilution that had a suitable level of precision and relative accuracy.

The antibodies and vitality stain utilized for ATM-3343 are listed in Table 6. The average of 6 measurements (one per occasion, per operator) was used for comparison for each sample type.

TABLE 6
ATM-3343 antibodies

| | |
|---|---|
| CD3 APC | Biolegend; 344812 |
| IgG1 APC | Biolegend; 400120 |

The range of the procedure was determined from the dilutional linearity exercise. The lower limit of quantitation was determined from the linearity exercise as the lowest dilution that had a suitable level of precision and relative accuracy.

Linearity: Linear regression analysis was performed to determine the R2 value by fitting the measured relative percentage of all assay runs against the nominal relative percentage as the fixed effect in a linear mixed model using JMP. The recovery for each level of the test sample from the linearity evaluation was compared to the nominal relative percentages in a linear mixed model with interaction of occasion and operator set as random effects using JMP.

Precision (Inter-assay variation): The variance estimate for intermediate precision was calculated by fitting the relative percentage into a linear mixed mode in JMP with occasion and operator set as random effects, and the nominal relative percentage as a fixed effect.

The range of the analytical procedure was determined from the accuracy and precision analysis using the linear mixed model generated using JMP. The lower limit of quantitation was determined from the accuracy and precision analysis using the linear mixed model generated using JMP. The LOQ was determined as the lowest sample concentration that met conditions of suitable accuracy and precision.

For the method qualification no acceptance criteria was established, but target criteria was listed in Table 7.

TABLE 7
Summary of Qualification Data Reporting and Target Criteria

| Qualification Parameter | Results Reported and Target Criteria |
|---|---|
| Linearity | R² = >0.8 |
| Accuracy/Trueness | Each dilution level: |
| | 100 ± 30% |
| Precision | Percent CV: |
| | Intermediate Precision: <20% |
| Range | Report Results |
| LOQ | Report Results |

### Example 3. CD3+ T Cell Quantification Assay for K-NK Drug Product

Purpose: This example reported the results of ATM-3343 method qualification, wherein accuracy, precision, linearity, limit of quantitation and range were tested. The qualification parameters and procedures were set forth in Example 2. These procedures were designed to qualify the sensitive analytical outcomes from ATM-3343 and define the limit of quantitation of % CD3+ residual T cell impurity in the K-NK DP. This example also summarized the method qualification data generated by two operators over three occasions as prescribed by Example 2. The results of the regression analysis comparing observed and predicted target measures are utilized to define the outcomes for Example 2 qualification parameters.

Materials and Reagents: The materials and reagents described in ATM-3343 were utilized for the execution of the qualification protocol as detailed in Example 2. The materials and reagents used are listed in Table 8.

TABLE 8
Reagents and Disposables

| Reagent/Disposables | Vendor/Part Number |
|---|---|
| Cell Staining Buffer | Biolegend; 420201 |
| Human Trustain FcX | Biolegend: 422302 |
| CD3 APC | Biolegend; 344812 |
| IgG1 APC | Biolegend; 400120 |
| Purified anti-human CD3 | Biolegend; 344802 |
| Propidium Iodide (PI) | Biolegend; 421301 |
| 5 mL FACS tube | VWR; 60819-295, or equivalent |
| 15 mL tubes | VWR; 76176-950, or equivalent |
| 50 mL tubes | VWR; 76176-952, or equivalent |
| Pipette tips, 200 μL | Rainin; RT-L200F, or equivalent |
| Pipette tips, 1000 μL | Rainin; RT-L1000F, or equivalent |
| Pipette tips, 20 μL | Rainin; RT-L10F, or equivalent |

Equipment used included Classic Attune™ Acoustic Focusing Cytometer, and Centrifuge (Thermo Scientific) (. Software used was Attune Cytometric Software v2.1 and JMP v. 14.3.0.

The cryopreserved PBMC-225398 samples were utilized as positive control to verify CD3+ gating and to set CD3+ gate for flow cytometry analysis. The preparation of PBMC-225398 is described in Example 2.

Test Article Material: The cryopreserved Batch 20035 d14 NK cells were utilized as test articles for this method qualification. The preparation of 20035 d14 cells for this method qualification was described in Example 2. The 20035 d14 cells were expanded by PM21 particles following CD3+ depletion and utilized in a standardized sample matrix in this assay, therefore these cells are representative of K-NK DP. Batch 20035 d14 has a mean % CD3+ content of 1.389% which was determined as part of method performance testing using a draft version of ATM-3343 (test article analyzed on 2 occasions, with 2 sample vials, and 2 replicates for n=8). 1.389% CD3+ is the expected (nominal) value for the test article.

arrows from top to lower table section) and subsequently included in the accompanying statistical analysis.

Procedure: The method qualification to test linearity, accuracy/trueness and precision were described in Example 2. Briefly, the cryo-recovered NK 20035 d14 samples were stained with Isotype-APC and CD3-APC antibodies as per ATM-3343. The isotype stained cells were also mock stained to prepare sample matrix diluent for CD3+ T cell spiking. These mock stained cells were treated with unlabeled purified CD3 antibody to block any CD3-APC binding due to any carryover antibody solution during CD3+ T cell spiking. Therefore, the serial dilutions for CD3+ T cell linearity estimation were prepared in sample matrix of K-NK DP and as per the requirements set in example 2 (FIG. 6). The data analysis was performed in JMP v14.3.0 statistical analysis software. The Nominal (Predicted) and Estimated (Results) % CD3+ values were collated from the reports of two analysts over three occasions each. The test article used for spiking required analysis by ATM-3343 to determine the CD3+ content for spiking mock stained preparations and a measurement was generated on each occasion. Therefore 6 measurements were performed according to the test method, these data points were included in the method qualification data analysis as an additional Level (Level 0) used to assess linearity, accuracy, precision and range. The residual plots of untransformed and Log (Log10, Log, Logist, Logit percentage and Logit) transformed Nominal CD3% and Estimated CD3% were examined to select regression model (FIG. 7). In the residual plot analysis, the untransformed data showed biased on the spreading of data with small spreading in lower level and wider spreading at higher level. Therefore, data was transformed for further analysis. The Log10 transformation was the simplest transformation that results in improved spreading of data in the residual plot. Therefore, Log10 transformed CD3% “Nominal” and “Estimated” data with three decimal places were selected for further analysis. Results reported in Tables were provided to three decimal places.

TABLE 9
Nominal (predicted) and Result (Estimated) % CD3+
data from two operators and three occasions each.

| | | | | | Log10 |
|---|---|---|---|---|---|
| Nominal | Log Nominal | | | Result | Result CD3% |
| CD3% (%) | CD3% (Log10%) | Operator | Occasion | CD3% (%) | (Log10%) |
| 1.000 | 0.000 | A | 1 | 1.052 | 0.022 |
| 1.000 | 0.000 | A | 2 | 1.028 | 0.012 |
| 1.000 | 0.000 | A | 3 | 0.989 | −0.005 |
| 1.000 | 0.000 | B | 4 | 1.019 | 0.008 |
| 1.000 | 0.000 | B | 5 | 0.977 | −0.010 |
| 1.000 | 0.000 | B | 6 | 1.130 | 0.053 |
| 0.250 | −0.602 | A | 1 | 0.271 | −0.567 |
| 0.250 | −0.602 | A | 2 | 0.262 | −0.582 |
| 0.250 | −0.602 | A | 3 | 0.266 | −0.575 |
| 0.250 | −0.602 | B | 4 | 0.246 | −0.609 |
| 0.250 | −0.602 | B | 5 | 0.236 | −0.627 |
| 0.250 | −0.602 | B | 6 | 0.266 | −0.575 |
| 0.063 | −1.201 | A | 1 | 0.068 | −1.167 |
| 0.063 | −1.201 | A | 2 | 0.063 | −1.201 |
| 0.063 | −1.201 | A | 3 | 0.070 | −1.155 |
| 0.063 | −1.201 | B | 4 | 0.067 | −1.174 |
| 0.063 | −1.201 | B | 5 | 0.060 | −1.222 |
| 0.063 | −1.201 | B | 6 | 0.063 | −1.201 |
| 0.016 | −1.796 | A | 1 | 0.019 | −1.721 |
| 0.016 | −1.796 | A | 2 | 0.020 | −1.699 |
| 0.016 | −1.796 | A | 3 | 0.019 | −1.721 |
| 0.016 | −1.796 | B | 4 | 0.018 | −1.745 |
| 0.016 | −1.796 | B | 5 | 0.017 | −1.770 |
| 0.016 | −1.796 | B | 6 | 0.018 | −1.745 |
| 0.004 | −2.398 | A | 1 | 0.005 | −2.301 |
| 0.004 | −2.398 | A | 2 | 0.005 | −2.301 |
| 0.004 | −2.398 | A | 3 | 0.007 | −2.155 |
| 0.004 | −2.398 | B | 4 | 0.005 | −2.301 |
| 0.004 | −2.398 | B | 5 | 0.005 | −2.301 |
| 0.004 | −2.398 | B | 6 | 0.006 | −2.222 |
| 1.389 | 0.143 | A | 1 | 1.382 | 0.141 |
| 1.389 | 0.143 | A | 2 | 1.424 | 0.154 |
| 1.389 | 0.143 | A | 3 | 1.381 | 0.140 |
| 1.389 | 0.143 | B | 4 | 1.546 | 0.189 |
| 1.389 | 0.143 | B | 5 | 1.421 | 0.153 |
| 1.389 | 0.143 | B | 6 | 1.505 | 0.178 |

Results: The accuracy was analyzed by using a linear mixed model by using “Operator” and “Occasion” as random factors. The recovery was calculated as per the equation below:

$$Recovery \% = ( \frac{{10}^{Log Estimated CD 3 \%}}{{10}^{Log Nominal CD 3 \%}} ) \times 100$$

The relative accuracy acceptance target criteria set forth in example 2 was 100±30%. The % recoveries for Nominal CD3% dilution levels, 0.016%-1.389% were within 100±30% acceptance criteria. The Nominal CD3% at 0.004% CD3+ T cell level did not meet the accuracy acceptance criteria (Table 10 and FIG. 8).

TABLE 10
Summary of recovery calculated for accuracy/trueness estimations.
Accuracy/Trueness

| Log Nominal | Log Estimate | Nominal | |
|---|---|---|---|
| CD3% | CD3% | CD3% | Recovery |
| (log %) | (log %) | (%) | (%) |
| −2.398 | −2.264 | 0.004 | 136.301 |
| −1.796 | −1.734 | 0.016 | 115.478 |
| −1.201 | −1.187 | 0.063 | 103.355 |
| −0.602 | −0.589 | 0.250 | 102.999 |
| 0.000 | 0.013 | 1.000 | 103.118 |
| 0.143 | 0.159 | 1.389 | 103.793 |

Precision: The precision was analyzed using a linear mixed model in the JMP v14.3.0 by using “Operator” and “Occasion” as random factors. As prescribed in Example 2, due to time limitations, the sequential repeat measurements of same samples were not acquired. Since the Level 5 (nominal 0.004% CD3+ T cells) did not reach the accuracy/trueness acceptance criteria, the nominal and estimated data from this level were excluded from precision analysis. The acceptance criteria set forth in Example 2 was to report % CV for precision and <20% CV for Intermediate Precision. The % CV results for “Operator”, “Occasion”, “Residual” and Intermediate Precision were 0.288, 2.386, 4.819 and 5.386% respectively (Table 11). Therefore, the Levels 0-4 (1.389%-0.016% CD3+ T cells) met the acceptance criteria for Precision analysis.

TABLE 11
Precision and Intermediate Precision analysis results

| Parameters | Var Component | % CV |
|---|---|---|
| Operator | 0.0000016 | 0.288 |
| Occasion | 0.0001074 | 2.386 |
| Residual (Repeatability) | 0.0004375 | 4.819 |
| Total (intermediate Precision) | 0.0005464 | 5.386 |

Linearity: To determine linearity of CD3+ quantitation, the CD3-APC stained cells were spiked and serially diluted in K-NK DP sample matrix as shown in FIG. 5 to predict five levels of % CD3+ T cell estimation (Levels 1-5 or 1.000%-0.004% CD3+ T cells). The sample matrix consisted of mock cells treated with Isotype-APC and purified unlabeled human CD3 antibody (negative stain). The unlabeled (unconjugated) CD3 antibody was included to block any CD3 staining due to carryover of CD3-APC antibody from the spiking sample into the sample matrix diluent. Undilute test article data generated for spiking was included as an additional level (Level 0) for evaluation of linearity; therefore, Linearity was assessed from 1.389%-0.004% CD3+ T cells. Since the estimated results from nominal Level 5 (0.004% CD3+ T cells) did not meet the accuracy acceptance criteria, this level was excluded from the linearity analysis in regression model. The regression plot and residual by predicted plot are shown in (FIG. 9). The Linearity target criteria set forth in example 2 was R2>0.8. The R² observed in the regression analysis was 0.999 (Table 12). Therefore, the linearity of Levels 0-4, 1.389%-0.016% CD3+ T cells, met the target acceptance criteria.

TABLE 12
Summary of regression model.
Summary of Fit

| | |
|---|---|
| RSquare | 0.999 |
| RSquare Adj | 0.999 |
| Root Mean Square Error | 0.026 |
| Mean of Response | −0.667 |
| Observations (or Sum Wgts) | 30.000 |

Range: The range for the % CD3+ T cell quantitation was determined from the accuracy and precision analysis using the linear mixed model generated in JMP v14.3.0. The accuracy was analyzed for five dilution levels (1-5) with expected 1.000%-0.004% CD3+ T cells in the four-fold dilution scheme within K-NK DP sample matrix. Inclusion of the undilute test article added an extra level, increasing the upper range to 1.389% CD3+ T cells. The predicted % CD3+ T cell quantitation for Levels (0-4), 1.389%-0.016% CD3+ T cells, met the accuracy acceptance criteria (100%±30%). The Level 5 (0.004% CD3+ T cells) did not meet the accuracy acceptance criteria. Therefore, the range of this method was defined as 1.389% to 0.016% CD3+ T cell quantitation. The % CD3+ T cells below this quantitation range should be reported as BLOQ (Below Limit of Quantitation), while % CD3+ T cells above this quantitation range should be reported as above ULOQ (Upper Limit of Quantitation). The assay validity criteria of acquiring 1×106 viable single cells must be met to report results within this quantitation range.

The K-NK DP consisted of NK cells that were expanded to high densities in-vitro, from a CD3+ T cell depleted PBMC population using PM21 particles. An impurity in the K-NK DP could be residual CD3+ T cells post depletion that may persist at levels lower than the defined limits of previously established analytical assays. Therefore, a Flow Cytometry-based ATM-3343 was developed to detect low (<0.3% CD3+ T cells) levels of residual CD3+ T cells as a potential impurity in K-NK DP. The ATM-3343 couples blocking and washing steps to specifically stain and acquire CD3+ T cells. This method utilized human TruStain FcX, CD3-APC and Isotype-APC reagents for specific detection and quantitation of % CD3+ T cells in K-NK DP. A reference PBMC population was utilized as positive control. Given the requirements of low levels of CD3+ quantitation, the ATM-3343 prescribed acquiring 1×106 viable single events from K-NK DP samples to satisfy statistical expectations.

data for qualification parameters including accuracy, precision, linearity, and range was generated by two operators over three occasions each. At each occasion, the Reference Material and the Isotype-APC or CD3-APC stained cells were acquired in addition to acquiring the five levels of predefined % CD3+ concentrations (Table 8). At each occasion, these dilution levels were prepared by spiking CD3+ T cells in mock (isotype and unlabeled CD3 treated) sample matrix of K-NK DP cells. The Flow Cytometry acquisition parameters were set to acquire 1×106 viable single cells from each sample based on sequential gating strategy (Total Events>MNC>Single Cells>Viable Cells>CD3+) set forth in ATM-3343 and Example 2.

TABLE 13
Execution of ATM-3343 Qualification Example 2
Overview of ATM-3343 Method Qualification (TD14455-0) Protocol Execution

| Operator | Occasion 1 | Occasion 2 | Occasion 3 | Results |
|---|---|---|---|---|
| A | 2 Sep. 2020 | 11 Sep. 2020 | 14 Sep. 2020 | |
| | RM-PBMC | RM-PBMC | RM-PBMC | 3 |
| | Isotype-APC | Isotype-APC | Isotype-APC | 3 |
| | CD3-APC (Spike) | CD3-APC (Spike) | CD3-APC (Spike) | 3 |
| | Mock (Diluent) | Mock (Diluent) | Mock (Diluent) | 3 |
| | Dilution Levels: % CD3+ T Cells | | | |
| | Dilution Level 5: 0.004% | Dilution Level 5: 0.004% | Dilution Level 5: 0.004% | 3 |
| | Dilution Level 4: 0.016% | Dilution Level 4: 0.016% | Dilution Level 4: 0.016% | 3 |
| | Dilution Level 3: 0.063% | Dilution Level 3: 0.063% | Dilution Level 3: 0.063% | 3 |
| | Dilution Level 2: 0.250% | Dilution Level 2: 0.250% | Dilution Level 2: 0.250% | 3 |
| | Dilution Level 1: 1.000% | Dilution Level 1: 1.000% | Dilution Level 1: 1.000% | 3 |
| B | 1 Sep. 2020 | 3 Sep. 2020 | 8 Sep. 2020 | |
| | RM-PBMC | RM-PBMC | RM-PBMC | 3 |
| | Isotype-APC | Isotype-APC | Isotype-APC | 3 |
| | CD3-APC (Spike) | CD3-APC (Spike) | CD3-APC (Spike) | 3 |
| | Mock (Diluent) | Mock (Diluent) | Mock (Diluent) | 3 |
| | Dilution Levels: % CD3+ T Cells | | | |
| | Dilution Level 5: 0.004% | Dilution Level 5: 0.004% | Dilution Level 5: 0.004% | 3 |
| | Dilution Level 4: 0.016% | Dilution Level 4: 0.016% | Dilution Level 4: 0.016% | 3 |
| | Dilution Level 3: 0.063% | Dilution Level 3: 0.063% | Dilution Level 3: 0.063% | 3 |
| | Dilution Level 2: 0.250% | Dilution Level 2: 0.250% | Dilution Level 2: 0.250% | 3 |
| | Dilution Level 1: 1.000% | Dilution Level 1: 1.000% | Dilution Level 1: 1.000% | 3 |

Summary: This method qualification was executed according to the procedures described Example 2, “Method Qualification Protocol for ATM-3343 T Cell Quantitation”. The K-NK drug product (DP) was comprised of NK cells expanded to high densities in-vitro from a CD3+ T cell depleted donor PBMC population. The ATM-3343 was developed to detect and quantify a low frequency of residual CD3+ T cells within a population of 1×106 viable single cells by Flow Cytometry. The data evaluated in this method qualification report was acquired by two analysts over three occasions each. The evaluation of the accuracy, precision and linearity parameters set forth by example 2 demonstrate suitability of the ATM-3343 for its intended use to detect and quantitate residual CD3+ T cells in K-NK DP. The ATM-3343 was accurate and precise to measure different levels within 0.016% to 1.389% range of CD3+ T cells impurity in the K-NK DP (Table 14). The R² value of 0.999 for % CD3+ quantitation in this range met the K-NK NP sample matrix linearity acceptance criteria. Thereby, this method establishes detecting 0.016% CD3+ T cells as the Limit of Quantitation (LOQ). The detection of CD3+ T cells by ATM-3343 is specific and reliably distinguishes positive events from the background noise of the isotype staining. The ATM-3343 must acquire 1×106 viable single events from K-NK DP to meet the validity criteria. Overall, the protocol used is suitable for the determination of CD3+ T cell impurity in the K-NK DP. The % CD3+ T cells below or above the quantitation range of 0.016% to 1.389% should be reported as BLOQ (Below Limit of Quantitation) or above ULOQ (Upper Limit of Quantitation), respectively. This quantitation range, reported from the acquisition of 1×106 viable single events, determined in this qualification can be set as a validity and system suitability criteria.

TABLE 14
Method Qualification Summary
Summary

| | | | |
|---|---|---|---|
| Qualification | | | |
| Parameter | Target Criteria | Result | Outcome |

Accuracy (% CD3+ Measurements)

| | | | |
|---|---|---|---|
| Level 0: 1.389%* | 100 ± 30% | 103.793% | Pass |
| Level 1: 1.000% | | 103.118% | Pass |
| Level 2: 0.250% | | 102.999% | Pass |
| Level 3: 0.063% | | 103.355% | Pass |
| Level 4: 0.016% | | 115.478% | Pass |
| Level 5: 0.004% | | 136.301% | Fail |

Precision (% CV): Test Article (1.389%) to Level 4 (0.016%)

| | | | |
|---|---|---|---|
| Operator | | 0.288% | Pass |
| Occasion | | 2.386% | Pass |
| Repeatability | | 4.819% | Pass |
| (residual) | | | |
| Intermediate | <20% | 5.386% | Pass |
| Precision | | | |

Linearity (% CD3+ Measurements)

| | | | |
|---|---|---|---|
| Level 0: 1.389%* | R² ≥ 0.8 | 0.999 | Pass |
| Level 1: 1.000% | | | |
| Level 2: 0.250% | | | |
| Level 3: 0.063% | | | |
| Level 4: 0.016% | | | |
| Level 5: 0.004% | N/A | NA | NA |

Range

| | | | |
|---|---|---|---|
| Range | Report Results | 0.004%-1.389% | 0.016%-1.389% |

Limit of Quantitation (LOQ)

| | | | |
|---|---|---|---|
| Limit of | Report Results | 0.016% | LOQ = 0.016% |
| Quantitation | | | |
| Assay Validity | | | |
| Event Count | Acquire 1 × 10⁶ | | |
| | viable single | | |
| | events | | |

*Level 0: Undilute test article Batch 20035 d 14, also referred to as CD3-APC (Spike) in Table 5.

Conclusion: The ATM-3343 was qualified to quantitate low 0.016%) % CD3+ T cells in K-NK DP. The accuracy, precision and linearity of % CD3+ T cells quantitation range between 0.016% to 1.389% were determined acceptable as per the qualification parameters. The ATM-3343 assay validity and system suitability criteria were required to reliably measure the CD3+ T cell impurities in K-NK DP.

1. A method for validating measurements by a fluorescence-based analytical instrument of a stained target cell population in a test sample, the method comprising:
a. negatively staining or having negatively stained target cells in a first portion of a reference sample comprising target cells expressing a target cell marker, wherein negatively staining the target cells in the reference sample first portion in (a) comprises introducing to the reference sample first portion (i) a fluorescent dead cell exclusion dye, (ii) a non-specific antibody conjugated to a fluorochrome, and (iii) a specific antibody capable of specifically binding a target marker on the target cell;
b. positively staining or having positively stained target cells in a second portion of the reference sample, wherein positively staining the target cells in cells in the reference sample second portion in (b) comprises introducing to the reference sample second portion (i) the fluorescent dead cell exclusion dye, and (ii) the specific antibody conjugated to the fluorochrome;
c. running the reference sample first portion through the instrument to obtain a fluorescence measurement indicative of the target cell concentration in the reference sample first portion, and running the reference sample second portion through the instrument to obtain a fluorescence measurement indicative of the target cell concentration in the reference sample second portion;
d. based on the fluorescence measurements obtained in (c), diluting the positively stained target cells in the second portion of the reference sample with the negatively stained target cells in the first portion of the reference sample to obtain a dilution sample having a highest concentration of the target cells, from which is prepared dilution series comprising a plurality of dilution samples, each dilution sample having a nominal concentration of the target cells, each nominal cell concentration greater than the concentration of target cells indicated by the fluorescence measurement of the negatively stained first portion in (a), wherein the nominal concentration in each dilution sample differs from the nominal concentration in each of the remaining dilution samples, wherein the highest concentration of the target cells is 2%, 1%, 0.5%, 0.2%, 0.1%, 0.05%, or 0.02%;
e. running the series of dilution samples from (d) through the instrument to obtain a series of fluorescence measurements comprising a fluorescence measurement for each dilution sample;
f. for each dilution sample, comparing the nominal cell concentration from (d) and the fluorescence measurement from (e) to quantify the performance of the staining method in the instrument, wherein comparing in (f) comprises performing a statistical calculation on the difference between the nominal cell concentration and the fluorescence measurement for each dilution sample to determine at least one of linearity, range, accuracy, precision, limit of detection (LOD) and lowest limit of quantification (LLOQ) for the instrument and staining method, wherein determining the LLOQ comprises identifying the concentration of target cells associated with a predetermined criterion for precision and a predetermined criterion for accuracy.

2-4. (canceled)

5. The method of claim 1, wherein the fluorescent dead cell exclusion dye is selected from a nucleic acid binding dye, propidium iodide, DAPI, DRAQ7, 7-AAD, TO-PRO-3, and an amine-reactive dye.

6. The method of claim 1, wherein the fluorochrome-conjugated non-specific antibody comprises an antibody lacking the capability of specifically binding to a cell antigen.

7. The method of claim 6, wherein the fluorochrome-conjugated non-specific antibody comprises any one of IgD, IgG, IgA, IgM or IgE conjugated to the fluorochrome.

8. The method of claim 1, wherein the fluorochrome is selected from Allophycocyanin (APC), APC C750, APC AF700, brilliant violet (BV)421, BV510, hilite 7 (H7) BV605, BV650, PE CF594, Fluorescein isothiocyanate (FITC), R-Phycoerythrin (PE or R-PE), PE-Cy7 (PE coupled to cyanine dye Cy7), APC-Cy7 (APC coupled to cyanine dye Cy7), APC-H7 (APC coupled to the Cy analogue Hilite 7 (H7)).

9. The method of claim 1, wherein the target cell marker is selected from CD3 as a T-cell marker, CD19 as a B-cell marker, CD235a as an erythrocyte marker, CD56 as a Natural Killer (NK) cell marker, CD14 as a monocyte marker, and CD66b as a granulocyte marker.

10. The method of claim 1, wherein the target cell population is a T cell population, and the target cell marker is CD3.

11. (canceled)

12. The method of claim 1, wherein the test sample comprises the target cell present in a concentration of no more than 2%, 1%, 0.5%, 0.2%, 0.1%, 0.05%, or 0.02%.

13. (canceled)

14. The method of claim 1, wherein the negatively stained first portion of the reference sample is prepared in the absence of a cell-depletion step.

15. The method of claim 1, wherein the fluorescence-based instrument is a flow cytometer.

16. A method for validating measurements by a flow cytometer of a stained target cell population in a test sample, the method comprising:
a. negatively staining or having negatively stained target cells in a first portion of a reference sample comprising target cells expressing a target cell marker, wherein negatively staining the target cells in the reference sample first portion in (a) comprises introducing to the reference sample first portion (i) a fluorescent dead cell exclusion dye, (ii) a non-specific antibody conjugated to a fluorochrome, and (iii) a specific antibody capable of specifically binding a target marker on the target cell, unconjugated to the fluorochrome;
b. positively staining or having positively stained target cells in a second portion of the reference sample, wherein positively staining the target cells in cells in the reference sample second portion in (b) comprises introducing to the reference sample second portion (i) the fluorescent dead cell exclusion dye, and (ii) the specific antibody conjugated to the fluorochrome;
c. running both the reference sample first portion and second portion through the flow cytometer to obtain fluorescence measurements indicative of the target cell concentration in each of the reference sample first portion and the reference sample second portion;
d. based on the fluorescence measurements obtained in (c), diluting the positively stained target cells in the second portion of the reference sample with the negatively stained target cells in the first portion of the reference sample to obtain a dilution sample having a highest concentration of the target cells, from which is prepared a series of dilution samples each having a nominal concentration of the target cells varying systematically across the dilution series, wherein the nominal cell concentration of at least one dilution sample is greater than the concentration of target cells indicated by the fluorescence measurement of the negatively stained first portion in (a), wherein the highest concentration of the target cells is 2%, 1%, 0.5%, 0.2%, 0.1%, 0.05%, or 0.02%;
e. running the series of dilution samples from (d) through the flow cytometer to obtain a series of fluorescence measurements comprising a fluorescence measurement for each a dilution sample;
f. for each dilution sample, comparing the nominal cell concentration from (d) and the fluorescence measurement from (e) to quantify the performance of the staining method in the flow cytometer, wherein comparing in (f) comprises performing a statistical calculation on the difference between the nominal cell concentration and the fluorescence measurement for each dilution sample to determine at least one of linearity, range, accuracy, precision, limit of detection (LOD), and lowest limit of quantification (LLOQ) for the instrument and staining method, and wherein determining the LLOQ comprises identifying the concentration of target cells associated with a predetermined criterion for precision and a predetermined criterion for accuracy.

17-19. (canceled)

20. The method of claim 16, wherein the fluorescent dead cell exclusion dye is selected from nucleic acid binding dye, propidium iodide, DAPI, DRAQ7, 7-AAD, TO-PRO-3, and an amine-reactive dye.

21. The method of claim 16, wherein the non-specific fluorochrome-conjugated antibody comprises an antibody lacking the capability of specifically binding to a cell antigen.

22. The method of claim 21, wherein the non-specific fluorochrome-conjugated antibody comprises any one of IgD, IgG, IgA, IgM or IgE conjugated to the fluorochrome.

23. The method of claim 16, wherein the fluorochrome is selected from Allophycocyanin (APC), APC C750, APC AF700, brilliant violet (BV)421, BV510, hilite 7 (H7) BV605, BV650, PE CF594, Fluorescein isothiocyanate (FITC), R-Phycoerythrin (PE or R-PE), PE-Cy7 (PE coupled to cyanine dye Cy7), APC-Cy7 (APC coupled to cyanine dye Cy7), APC-H7 (APC coupled to the Cy analogue Hilite 7 (H7)).

24. The method of claim 16, wherein the target cell marker is selected from CD3 as a T-cell marker, CD19 as a B-cell marker, CD235a as an erythrocyte marker, CD56 as a Natural Killer (NK) cell marker, CD14 as a monocyte marker, and CD66b as a granulocyte marker.

25. The method of claim 16, wherein the target cell population is a T cell population, and the target cell marker is CD3.

26-27. (canceled)

28. The method of claim 16, wherein the test sample comprises the target cell present in a concentration of no more than 2%, 1%, 0.5%, 0.2%, 0.1%, 0.05%, or 0.02%.

29-30. (canceled)

31. A non-transitory computer-readable medium comprising instructions for a computer processor for performing the comparing step of claim 1.

32. A system for validating fluorescence measurements in a test sample made by a fluorescence-based instrument, the system comprising: the fluorescence-based instrument; and a computer coupled with the fluorescence-based instrument and comprising the computer readable medium of claim 31, wherein the fluorescence-based instrument is a flow cytometer.

33-34. (canceled)
